SEQUENCE LISTING (20 sequences)

SEQ ID NO: 1 - protein, 1674 aa, Clostridium thermocellum
MRKVISMLLVVAMLTTIFAAMIPQTVSAATMTVEIGKVTAAVGSKVEIPITLKGVPSKGMANCDFVLGYDPNVLEVTEVKPGSIIKDPDPSKSFDSAIYPDRKMIVFLFAEDSGRGTYAITQDGVFATIVATVKSAAAAPITLLEVGAFADNDLVEISTTFVAGGVNLGSSVPTTQPNVPSDGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNATPTKGATPTNTATPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGVTSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQKTQFSDGGVNVGGTTVPTTPPASTTPTDDPNAIKIKVDTVNAKPGDTVNIPVRFSGIPSKGIANCDFVYSYDPNVLEIIEIKPGELIVDPNPDKSFDTAVYPDRKIIVFLLTEDSGTGAYAITKDGVFATIVAKVKSGAPNGLSVIKFVEVGGFANNDLVEQKTQFFDGGVNVGDTTVPTTPTTPVTTPTDDPNAVRIKVDTVNAKTGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTEPATPTTPVTTPTTTDGLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQRTQFFDGGVNVGDTTVPTTPTTPVTTPTDDSNAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTVPTTSPTTTPPEPTIAPNKLTLKIGRAEGRPGDTVEIPVNLYGVPQKGIASGDFVVSYDPNVLEIIEIEPGELIVDPNPTKSFDTAVYPDRKMIVFLFAEDSGTGAYAITEDGVFATIVAKVKEGAPEGFSAIEISEFGAFADNDLVEVETDLINGGVLVTNKTVIEGYKVSGYILPDFSFDATVAPLVKAGFKVEIVGTELYAVTDANGYFEITGVPANASGYTLKISRATYLDRVIANVVVTGDTSVSTSQAPIMMWVGDIVKDNSINLLDVAEVIRCFNATKGSANYVEELDINRNGAINMQDIMIVHKHFGATSSDY

SEQ ID NO: 2 - protein, 159 aa, Clostridium thermocellum
PVSGNLKVEFYNSNPSDTTNSINPQFKVTNTGSSAIDLSKLTLRYYYTVDGQKDQTFWCDHAAIIGSNGSYNGVTSNVKGTFVKMSSSTNNADTYLEISFTGGTLEPGAHVQIQGRFAKNDWSNYTQSNDYSFKSASQFVEWDQVTAYLNGVLVWGKEP

SEQ ID NO: 3 - protein, 419 aa, Unknown
  SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTAAIKLDDKDPNFKDQVILLNKHIDAYKTFPPTEPKKDKKKKADETQALPQRQKKQQTVTLLPAADLDDFSKQLQQSMSSADSTQA

SEQ ID NO: 4 - protein, 202 aa, Unknown
  SARS-CoV-2
MITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 5 - protein, 124 aa, Artificial Sequence
  Synthetic
AGPGANPPGTTTTSRPATTTGSSPGPQACSSVWGQCGGQNWSGPTCCASGSTCVYSNDYYSQCLPGANPPGTTTTSRPATTTGSSPGPTQSHYGQCGGIGYSGPTVCASGTTCQVLNPYYSQCL

SEQ ID NO: 6 - protein, 615 aa, Artificial Sequence
  Synthetic
MGSSHHHHHHSSGLVPRGSHMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTAAIKLDDKDPNFKDQVILLNKHIDAYKTFPPTEPKKDKKKKADETQALPQRQKKQQTVTLLPAADLDDFSKQLQQSMSSADSTQAGSGGGGSGGGGSGGGGSPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEP

SEQ ID NO: 7 - protein, 398 aa, Artificial Sequence
  Synthetic
MGSSHHHHHHSSGLVPRGSHMITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGSGGGGSGGGGSGGGGSPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEP

SEQ ID NO: 8 - protein, 1674 aa, Clostridium thermocellum
MRKVISMLLVVAMLTTIFAAMIPQTVSAATMTVEIGKVTAAVGSKVEIPITLKGVPSKGMANCDFVLGYDPNVLEVTEVKPGSIIKDPDPSKSFDSAIYPDRKMIVFLFAEDSGRGTYAITQDGVFATIVATVKSAAAAPITLLEVGAFADNDLVEISTTFVAGGVNLGSSVPTTQPNVPSDGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNATPTKGATPTNTATPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQKTQFSDGGVNVGGTTVPTTPPASTTPTDDPNAIKIKVDTVNAKPGDTVNIPVRFSGIPSKGIANCDFVYSYDPNVLEIIEIKPGELIVDPNPDKSFDTAVYPDRKIIVFLLTEDSGTGAYAITKDGVFATIVAKVKSGAPNGLSVIKFVEVGGFANNDLVEQKTQFFDGGVNVGDTTVPTTPTTPVTTPTDDPNAVRIKVDTVNAKTGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTEPATPTTPVTTPTTTDGLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQRTQFFDGGVNVGDTTVPTTPTTPVTTPTDDSNAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTVPTTSPTTTPPEPTIAPNKLTLKIGRAEGRPGDTVEIPVNLYGVPQKGIASGDFVVSYDPNVLEIIEIEPGELIVDPNPTKSFDTAVYPDRKMIVFLFAEDSGTGAYAITEDGVFATIVAKVKEGAPEGFSAIEISEFGAFADNDLVEVETDLINGGVLVTNKTVIEGYKVSGYILPDFSFDATVAPLVKAGFKVEIVGTELYAVTDANGYFEITGVPANASGYTLKISRATYLDRVIANVVVTGDTSVSTSQAPIMMWVGDIVKDNSINLLDVAEVIRCFNATKGSANYVEELDINRNGAINMQDIMIVHKHFGATSSDY

SEQ ID NO: 9 - protein, 159 aa, Clostridium thermocellum
PVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEP

SEQ ID NO: 10 - protein, 15 aa, Artificial Sequence
  Synthetic
GGGGSGGGGSGGGGS

SEQ ID NO: 11 - protein, 6 aa, Artificial Sequence
  Synthetic
GGGGGG

SEQ ID NO: 12 - protein, 8 aa, Artificial Sequence
  Synthetic
GGGGGGGG

SEQ ID NO: 13 - protein, 15 aa, Artificial Sequence
  Synthetic
EAAAKEAAAKEAAAK

SEQ ID NO: 14 - protein, 46 aa, Artificial Sequence
  Synthetic
AEAAAKEAAAKEAAAKEAAAKALEAEAAAKEAAAKEAAAKEAAAKA

SEQ ID NO: 15 - protein, 5 aa, Artificial Sequence
  Synthetic
PAPAP

SEQ ID NO: 16 - protein, 17 aa, Artificial Sequence
  Synthetic
VSQTSKLTRAETVFPDV

SEQ ID NO: 17 - protein, 6 aa, Artificial Sequence
  Synthetic
RVLAEA

SEQ ID NO: 18 - protein, 10 aa, Artificial Sequence
  Synthetic
EDVVCCSMSY

SEQ ID NO: 19 - protein, 8 aa, Artificial Sequence
  Synthetic
GGIEGRGS

SEQ ID NO: 20 - protein, 4 aa, Artificial Sequence
  Synthetic
GFLG

(19) United States
(12) Patent Application Publication
(10) Pub. No.: US 2023/0408515 A1
(43) Pub. Date: Dec. 21, 2023

(54) ENGINEERED PROTEIN FOR RAPID, EFFICIENT CAPTURE OF PATHOGEN-SPECIFIC ANTIBODIES

(71) Applicant: Massachusetts Institute of Technology, Cambridge, MA (US)

(72) Inventors: Hadley Sikes Johnson, Arlington, MA (US); Eric Alexander Miller, Somerville, MA (US); Seunghyeon Kim, Boston, MA (US)

(73) Assignee: Massachusetts Institute of Technology, Cambridge, MA (US)

(21) Appl. No.: 18/035,809

(22) PCT Filed: Nov. 10, 2021
(86) PCT No.: PCT/US2021/058726

§ 371 Date: May 8, 2023

Related U.S. Application Data
(60) Provisional application No. 63/111,781, filed on Nov. 10, 2020.

(51) Int. Cl. G01N 33/569; G01N 33/58; C07K 14/005

(52) U.S. Cl. CPC G01N 33/56983; G01N 33/581; C07K 14/005; G01N 2333/165; G01N 2469/20; G01N 2400/26; C12N 2770/20022; C07K 2319/20

(57) ABSTRACT

The present disclosure relates to proteins comprising a target-binding domain for detection of anti-SARS-CoV-2 antibodies, methods, compositions and kits thereof.

### RELATED APPLICATION

This application claims priority under 35 U.S.C. § 119(e) to U.S. provisional application No. 63/111,781, filed Nov. 10, 2020, the content of which is incorporated herein by reference in its entirety.

### FIELD

Engineered proteins that specifically bind pathogen-specific antibodies, and methods for use thereof, are disclosed herein.

### BACKGROUND

Severe acute respiratory syndrome coronavirus 2 (SARS-CoV-2) is a novel coronavirus and the causative agent of coronavirus disease 2019 (Covid-19). As an emerging threat to humans and other mammals, SARS-CoV-2 has presented an urgent challenge to doctors and public health officials worldwide.

### SUMMARY

Containing and minimizing the effects of SARS-CoV-2 requires generation of new diagnostic methods to specifically and efficiently detect the presence of antibodies that bind to SARS-CoV-2. Detecting antibodies to SARS-CoV-2 can allow doctors and public health officials to determine who has been exposed to SARS-CoV-2. Rapid, specific, economical testing for anti-SARS-CoV-2 antibodies is needed to combat a global health crisis. A paper-based diagnostic using recombinant proteins that bind anti-SARS-CoV-2 antibodies (antibodies that bind to SARS-CoV-2) linked to cellulose binding domains is described herein. The instant disclosure relates to recombinant proteins, in particular fusion proteins comprising an antigenic SARS-CoV-2 protein and a cellulose-binding domain (CBD), having the ability to bind anti-SARS-CoV-2 antibodies. These recombinant scaffold proteins may be used alone or in combination with domains capable of binding to paper, such as cellulose binding domains (CBD), to generate inexpensive, scalable diagnostics for rapid, specific detection of SARS-CoV-2.

In an aspect, the instant disclosure relates to a fusion protein comprising a cellulose binding domain (CBD) and a protein which binds antibodies to SARS-CoV-2. In some embodiments, the C-terminus of the antibody-binding protein is linked to the N-terminus of the CBD. In some embodiments, the antibody-binding protein is linked to the CBD through a linker; in some embodiments the linker is a Gly-Ser linker.

In some embodiments, the antibody-binding protein comprises a SARS-CoV-2 protein. In some embodiments, the antibody-binding protein comprises a SARS-CoV-2 nucleoprotein or a fragment thereof. In some embodiments, the antibody-binding protein comprises at least 85%, at least 90%, at least 95%, at least 99% or 100% sequence identity to the amino acid sequence of SEQ ID NO: 3. In some embodiments, the antibody-binding domain comprises a SARS-CoV-2 spike protein or a fragment thereof. In some embodiments, the antibody-binding protein comprises at least 85%, at least 90%, at least 95%, at least 99%, or 100% sequence identity of the amino acid sequence of SEQ ID NO: 4.

In some embodiments, the CBD is a type 3a CBD, or the type 1 dimerized CBD (dCBD). In some embodiments, the type 3a CBD is a domain of the protein CipA from Clostridium thermocellum. In some embodiments, the fusion protein sequence is SEQ ID NO: 13. In some embodiments, the fusion protein sequence is SEQ ID NO: 14.

In another aspect, the instant disclosure relates to a method for detecting an anti-SARS-CoV-2 antibody. In some embodiments, the method comprises: (a) contacting a fusion protein comprising a protein that binds an anti-SARS-CoV-2 antibody and a CBD with a cellulose-containing substrate for a time sufficient for the fusion protein to bind the cellulose-containing substrate; (b) contacting the fusion protein bound to the cellulose-containing substrate with a sample comprising or suspected to comprise an antibody that binds SARS-CoV-2; and (c) detecting the anti-SARS-CoV-2 antibody, if present, bound by the antibody-binding protein. In some embodiments, the method comprises: (a) contacting a fusion protein comprising a protein that binds an anti-SARS-CoV-2 antibody and a CBD with a sample comprising or suspected to comprise an anti-SARS-CoV-2 antibody, wherein the anti-SARS-CoV-2 antibody binds to the fusion protein and forms a complex; (b) contacting the complex with a cellulose-containing substrate for a time sufficient for the complex to bind to the cellulose-containing substrate; and (c) detecting the anti-SARS-CoV-2 antibody, if present, bound by the antibody-binding protein. In some embodiments, the instant disclosure relates to a method for assessing a presence or amount of an anti-SARS-CoV-2 antibody in a sample, comprising contacting the sample with one or more of the fusion protein described herein and measuring the presence or amount of the anti-SARS-CoV-2 antibody in the sample.

In some embodiments, the method further comprises rinsing the cellulose-containing substrate with a buffer solution before detecting the anti-SARS-CoV-2 antibody bound by the antibody-binding protein. In some embodiments, the method further comprises providing treatment to the subject.

In some embodiments, detecting comprises addition of a detectably-labeled antibody that binds to the anti-SARS-CoV-2 antibody. In some embodiments, the detectably-labeled antibody is an enzyme-labeled antibody. In some embodiments, the enzyme-labeled antibody binds to antibodies from the organism form which the sample being analyzed is obtained, such as by binding to a constant region of such antibodies. For example, if a human sample is analyzed, then any anti-SARS-CoV-2 antibody to be detected in that sample is from a human, and accordingly the enzyme-labeled antibody used to detect the anti-SARS-CoV-2 antibody binds to a human antibody. In some embodiments, the enzyme-labeled antibody binds to a human IgG or human IgM antibody. In some embodiments, the enzyme-labeled antibody is labeled with horseradish peroxidase (HRP).

In some embodiments, the fusion protein is in molar excess of the anti-SARS-CoV-2 antibody. In some embodiments, the fusion protein is in at least 10-fold molar excess of the anti-SARS-CoV-2 antibody.

In some embodiments, the anti-SARS-CoV-2 antibody is an anti-SARS-CoV-2 antibody that binds to SARS-CoV-2 nucleoprotein or SARS-CoV-2 spike protein. In some embodiments, at least 50% of the anti-SARS-CoV-2 antibody is bound by the fusion protein.

In some embodiments, the cellulose-containing substrate is paper, nitrocellulose, or cellulose powder. In some embodiments, the paper is chromatography paper. In some embodiments, the chromatography paper is unmodified.

In some embodiments, the sample is a biological sample from a subject. In some embodiments, the subject is a mammal. In some embodiments, the subject is a human.

In another aspect, the instant disclosure relates to a kit for assessing a presence or amount of an anti-SARS-CoV-2 antibody, wherein the kit comprises a container containing one or more of the fusion proteins described herein. In some embodiments, further comprising a cellulose-containing substrate. In some embodiments, the fusion protein is bound to the cellulose-containing substrate. In some embodiments, the fusion protein is not bound to the cellulose-containing substrate. In some embodiments, the cellulose-containing substrate is paper, nitrocellulose, or cellulose powder. In some embodiments, the paper is chromatography paper. In some embodiments, the chromatography paper is unmodified.

The invention is not limited in its application to the details of construction and the arrangement of components set forth in the following description or illustrated in the drawings. The invention is capable of other embodiments and of being practiced or of being carried out in various ways. Each of the above embodiments and aspects may be linked to any other embodiment or aspect. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 depicts the organization of an exemplary paper-based device for the detection of antibodies binding to SARS-CoV-2 N protein. Spots indicate zones where reagents and samples are applied. T indicates a spot where samples to be tested are added; C indicates a spot for a control reaction. M and G represent use of an IgM or IgG detection reagent.

FIGS. 2A-2D depict the results of an assay, such as the one shown in FIG. 1. FIG. 2A shows a positive result, indicating detection of anti-SARS-CoV-2 antibodies that bind SARS-CoV-2 N protein. FIG. 2B shows a positive result, indicating detection of anti-SARS-CoV-2 antibodies that bind SARS-CoV-2 N protein. FIG. 2C shows a negative result, indicating that anti-SARS-CoV-2 antibodies that bind SARS-CoV-2 N protein were not detected. FIG. 2D shows a negative result, indicating that anti-SARS-CoV-2 antibodies that bind SARS-CoV-2 N protein were not detected.

### DETAILED DESCRIPTION

Rapid, specific, economical testing for SARS-CoV-2 antibodies is needed to combat a global health crisis. A paper-based diagnostic using recombinant proteins that bind anti-SARS-CoV-2 antibodies (antibodies that bind to SARS-CoV-2) linked to cellulose binding domains is described herein. The instant disclosure relates to recombinant proteins, in particular fusion proteins comprising an antigenic SARS-CoV-2 protein and a cellulose-binding domain (CBD), having the ability to bind anti-SARS-CoV-2 antibodies. These recombinant scaffold proteins may be used alone or in combination with domains capable of binding to paper, such as cellulose binding domains (CBD), to generate inexpensive, scalable diagnostics for rapid, specific detection of SARS-CoV-2.

### Fusion Proteins

Domain that Binds SARS-CoV-2 Antibodies

In some aspects, provided herein is a fusion protein that incorporates a substrate-anchoring domain and a domain that binds an antibody that binds a SARS-CoV-2 antigen, such as an antibody that binds to SARS-CoV-2 nucleoprotein (N protein) or an antibody that binds to SARS-CoV-2 spike protein.

SARS-CoV-2 N protein and SARS-CoV-2 spike protein are components of the SARS-CoV-2 viral particle which are exposed such that a subject exposed to a SARS-CoV-2 viral particle may develop an immune response specific to all or a portion of SARS-CoV-2 N protein and/or spike protein.

SARS-CoV-2 N Protein Comprises the Sequence:

(SEQ ID NO: 3)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTAAIKLDDKDPNFKDQV
ILLNKHIDAYKTFPPTEPKKDKKKKADETQALPQRQKKQQTVTLLPAADL
DDFSKQLQQSMSSADSTQA

SARS-CoV-2 Spike Protein Comprises the Sequence:

(SEQ ID NO: 4)
MITNLCPFGEVENATRFASVYAWNRKRISNCVADYSVLYNSASFSTFKCY
GVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKS
TN

### Substrate-Anchoring Domain

Provided herein are proteins that bind anti-SARS-CoV-2 antibodies, which proteins are linked to a substrate-anchoring domain. In some embodiments, the substrate-anchoring domain is a CBD (cellulose binding domain) or a CBM (carbohydrate binding molecule).

In some embodiments, the CBM has carbohydrate-binding activity. In some embodiments, the CBM is CBM1, CBM2, CBM3, CBM4, CBM5, CBM6, CBM9, CBM10, CBM11, CBM12, CBM14, CBM15, CBM17, CBM18, CBM19, CBM20, CBM21, CBM25, CBM27, CBM28, CBM32, CBM33, CBM48, or CBM49. The nucleic acid and amino acid sequences of CBMs contemplated herein have been described, such as those disclosed in www.cazypedia.org/index.php/Carbohydrate-binding_modules, and can be readily identified by one of ordinary skill in the art using a BLAST search.

In some embodiments, the substrate-anchoring domain is a CBD. Orthologs of CBDs have been described in various species, including, but not limited to Micromonospora mirobrigensis (GenBank ID: SCF42127.1), Mycobacterium tuberculosis (GenBank ID: CNE10097.1), Micromonospora nigra (GenBank ID: SCL15442.1), Micromonospora mirobrigensis (GenBank ID: SCF04121.1), Cellulomonas Fimi (PDB: 1EXH_A), Mycobacterium kansasii 732 (GenBank: EUA13076.1), Ruminococcus albus 8 (GenBank: EGC02462.1), Leifsonia aquatic (NCBI Reference Sequence: WP_021763186.1), Schizosaccharomyces pombe (NCBI Reference Sequence: NP_593986.1), Desulfitobacterium hafniense (GenBank: CDX04743.1). CBDs expressed in other species that are known to one of ordinary skill in the art, such as CBDs of families I, II, III and IV disclosed, for instance, in Tomme et al., J Chromatogr B Biomed Sci Appl (1998) 715(1):283-96, are also contemplated herein.

Different types of CBDs are also contemplated herein. In some embodiments, a type 1 CBD is contemplated herein and serves as the substrate-anchoring domain of a fusion protein described herein. In some embodiments, the type 1 CBD comprises or consists of SEQ ID NO: 5.

Amino Acid Sequence of Type 1 CBD (SEQ ID NO: 5):

(SEQ ID NO: 5)
AGPGANPPGTTTTSRPATTTGSSPGPQACSSVWGQCGGQNWSGPTCCASG
STCVYSNDYYSQCLPGANPPGTTTTSRPATTTGSSPGPTQSHYGQCGGIG
YSGPTVCASGTTCQVLNPYYSQCL

Orthologs of type 1 CBDs have been described in various species, including, but not limited to Trichoderma reesei QM6a (NCBI Reference Sequence: XP_006969224.1); Rhizopus oryzae (GenBank: BAC53988.1); Schizosaccharomyces japonicus yFS275 (NCBI Reference Sequence: XP_002172247.1); Trichoderma virens Gv29-8 (NCBI Reference Sequence: XP_013954979.1); Trichoderma viride (GenBank: CAA37878.1) are also contemplated herein. Type 1 CBDs or orthologs thereof in other species known to one of ordinary skill in the art are also contemplated herein.

In some embodiments, a type 3a CBD is contemplated herein and serves as the substrate-anchoring domain of a fusion protein described herein. In some embodiments, the type 3a CBD is a domain of the CipA protein from Clostridium thermocellum (Genbank: HF912725.1; UniProtKB/TrEMBL: N1JW75)

Amino Acid Sequence of CipA Protein from Clostridium thermocellum (SEQ ID NO: 1):

(SEQ ID NO: 1)

MRKVISMLLV VAMLTTIFAA MIPQTVSAAT MTVEIGKVTA
AVGSKVEIPI MRKVISMLLV VAMLTTIFAA MIPQTVSAAT
MTVEIGKVTA AVGSKVEIPI TLKGVPSKGM ANCDFVLGYD
PNVLEVTEVK PGSIIKDPDP SKSFDSAIYP DRKMIVELFA
EDSGRGTYAI TQDGVFATIV ATVKSAAAAP ITLLEVGAFA
DNDLVEISTT FVAGGVNLGS SVPTTQPNVP SDGVVVEIGK
VTGSVGTTVE IPVYFRGVPS KGIANCDFVF RYDPNVLEII
GIDPGDIIVD PNPTKSFDTA IYPDRKIIVF LFAEDSGIGA
YAITKDGVFA KIRATVKSSA PGYITFDEVG GFADNDLVEQ
KVSFIDGGVN VGNATPTKGA TPTNTATPTK SATATPTRPS
VPTNTPTNTP ANTPVSGNLK VEFYNSNPSD TINSINPQFK
VINTGSSAID LSKLTLRYYY TVDGQKDGTF WCDHAAIIGS
NGSYNGVTSN VKGTFVKMSS STNNADTYLE ISFTGGTLEP
GAHVGIQGRF AKNDWSNYTQ SNDYSFKSAS QFVEWDQVTA
YLNGVLVWGK EPGGSVVPST QPVTTPPATT KPPATTIPPS
DDPNAIKIKV DTVNAKPGDT VNIPVRFSGI PSKGIANCDF
VYSYDPNVLE IIEIKPGELI VDPNPDKSFD TAVYPDRKII
VFLFAEDSGT GAYAITKDGV FATIVAKVKS GAPNGLSVIK
FVEVGGFANN DLVEQKTQFS DGGVNVGGIT VPTTPPASTT
PTDDPNAIKI KVDTVNAKPG DTVNIPVRES GIPSKGIANC
DFVYSYDPNV LEIIEIKPGE LIVDPNPDKS FDTAVYPDRK
IIVFLLTEDS GIGAYAITKD GVFATIVAKV KSGAPNGLSV
IKFVEVGGFA NNDLVEQKTG FFDGGVNVGD TTVPTTPTTP
VTTPTDDPNA VRIKVDTVNA KTGDTVRIPV RFSGIPSKGI
ANCDFVYSYD PNVLEIIEIE PGDIIVDPNP DKSFDTAVYP
DRKIIVFLFA EDSGTGAYAI TKDGVFATIV AKVKSGAPNG
LSVIKFVEVG GFANNDLVEQ KTQFFDGGVN VGDTTEPATP
TTPVITPTTT DGLDAVRIKV DTVNAKPGDI VRIPVRFSGI
PSKGIANCDF VYSYDPNVLE IIEIEPGDII VDPNPDKSFD
TAVYPDRKII VFLFAEDSGT GAYAITKDGV FATIVAKVKS
GAPNGLSVIK FVEVGGFANN DLVEQRTGFF DGGVNVGDTT
VPTTPTTPVT TPTDDSNAVR IKVDTVNAKP GDTVRIPVRF
SGIPSKGIAN CDFVYSYDPN VLEIIEIEPG DIIVDPNPDK
SFDTAVYPDR KIIVFLFAED SGTGAYAITK DGVFATIVAK
VKSGAPNGLS VIKFVEVGGF ANNDLVEQKT QFFDGGVNVG
DTTVPTTSPT TTPPEPTIAP NKLILKIGRA EGRPGDIVEI
PVNLYGVPQK GIASGDFVVS YDPNVLEIIE IEPGELIVDP
NPTKSFDTAV YPDRKMIVEL FAEDSGIGAY AITEDGVFAT
IVAKVKEGAP EGFSAIEISE FGAFADNDLV EVETDLINGG
VLVINKTVIE GYKVSGYILP DFSFDATVAP LVKAGFKVEI
VGTELYAVID ANGYFEITGV PANASGYTLK ISRATYLDRV
IANVVVTGDT SVSTSQAPIM MWVGDIVKDN SINLLDVAEV
IRCFNATKGS ANYVEELDIN RNGAINMQDI MIVHKHFGAT
SSDY

In some embodiments, the underlined valine (V) residue of SEQ ID NO: 1 is an isoleucine (I), which corresponds to SEQ ID NO: 8.

Amino Acid Sequence of CipA Protein from Clostridium thermocellum with an Isoleucine in Lace of a Valine (SEQ ID NO:8):

(SEQ ID NO: 8)

MRKVISMLLV VAMLITIFAA MIPQTVSAAT MTVEIGKVTA
AVGSKVEIPI MRKVISMLLV VAMLITIFAA MIPQTVSAAT
MTVEIGKVTA AVGSKVEIPI TLKGVPSKGM ANCDFVLGYD
PNVLEVTEVK PGSIIKDPDP SKSFDSAIYP DRKMIVFLFA
EDSGRGTYAI TQDGVFATIV ATVKSAAAAP ITLLEVGAFA
DNDLVEISTT FVAGGVNLGS SVPTTQPNVP SDGVVVEIGK
VTGSVGTTVE IPVYFRGVPS KGIANCDFVF RYDPNVLEII
GIDPGDIIVD PNPTKSFDTA IYPDRKIIVF LFAEDSGIGA
YAITKDGVFA KIRATVKSSA PGYITFDEVG GFADNDLVEQ
KVSFIDGGVN VGNATPTKGA TPTNTATPTK SATATPTRPS
VPTNTPTNTP ANTPVSGNLK VEFYNSNPSD TINSINPQFK
VINTGSSAID LSKLTLRYYY TVDGQKDOTF WCDHAAIIGS
NGSYNGITSN VKGTFVKMSS STNNADTYLE ISFTGGTLEP
GAHVOIQGRF AKNDWSNYTQ SNDYSFKSAS QFVEWDQVTA
YLNGVLVWGK EPGGSVVPST QPVTTPPATT KPPATTIPPS
DDPNAIKIKV DTVNAKPGDT VNIPVRFSGI PSKGIANCDF
VYSYDPNVLE IIEIKPGELI VDPNPDKSFD TAVYPDRKII
VFLFAEDSGT GAYAITKDGV FATIVAKVKS GAPNGLSVIK
FVEVGGFANN DLVEQKTOFS DGGVNVGGTT VPTTPPASTT
PTDDPNAIKI KVDTVNAKPG DTVNIPVRES GIPSKGIANC
DFVYSYDPNV LEIIEIKPGE LIVDPNPDKS FDTAVYPDRK
IIVFLLTEDS GIGAYAITKD GVFATIVAKV KSGAPNGLSV
IKFVEVGGFA NNDLVEQKTQ FFDGGVNVGD TTVPTTPTTP
VITPTDDPNA VRIKVDIVNA KIGDIVRIPV RFSGIPSKGI
ANCDFVYSYD PNVLEIIEIE PGDIIVDPNP DKSFDTAVYP
DRKIIVFLFA EDSGIGAYAI TKDGVFATIV AKVKSGAPNG
LSVIKFVEVG GFANNDLVEQ KTQFFDGGVN VGDTTEPATP
TTPVTTPTTT DGLDAVRIKV DTVNAKPGDI VRIPVRFSGI
PSKGIANCDF VYSYDPNVLE IIEIEPGDII VDPNPDKSFD
TAVYPDRKII VFLFAEDSGT GAYAITKDGV FATIVAKVKS
GAPNGLSVIK FVEVGGFANN DLVEQRTQFF DGGVNVGDTT
VPTTPTTPVT TPTDDSNAVR IKVDTVNAKP GDTVRIPVRF
SGIPSKGIAN CDFVYSYDPN VLEIIEIEPG DIIVDPNPDK
SFDTAVYPDR KIIVFLFAED SGTGAYAITK DGVFATIVAK
VKSGAPNGLS VIKFVEVGGF ANNDLVEQKT QFFDGGVNVG
DITVPTISPT TTPPEPTIAP NKLILKIGRA EGRPGDIVEI
PVNLYGVPQK GIASGDFVVS YDPNVLEIIE IEPGELIVDP
NPTKSFDTAV YPDRKMIVEL FAEDSGTGAY AITEDGVFAT
IVAKVKEGAP EGFSAIEISE FGAFADNDLV EVETDLINGG
VLVINKTVIE GYKVSGYILP DFSFDATVAP LVKAGFKVEI
VGTELYAVID ANGYFEITGV PANASGYTLK ISRATYLDRV
IANVVVTGDT SVSTSQAPIM MWVGDIVKDN SINLLDVAEV
IRCFNATKGS ANYVEELDIN RNGAINMQDI MIVHKHFGAT
SSDY

The amino acid sequence of the type 3a CBD of CipA protein from Clostridium thermocellum, which corresponds to amino acids 364-522 of the CipA protein from Clostridium thermocellum corresponds to SE ID NO: 2.

(SEQ ID NO: 2)

PVSGNLK VEFYNSNPSD TINSINPQFK VINTGSSAID
LSKLTLRYYY TVDGQKDQTF WCDHAAIIGS NGSYNGVTSN
VKGIFVKMSS SINNADTYLE ISFTGGTLEP GAHVQIQGRF
AKNDWSNYTQ SNDYSFKSAS QFVEWDQVTA YLNGVLVWGK
EP

In some embodiments, the underlined valine (V) residue of SEQ ID NO: 2 is an isoleucine (I), which corresponds to SEQ ID NO: 9.

(SEQ ID NO: 9)

PVSGNLK VEFYNSNPSD TINSINPQFK VINTGSSAID
LSKLTLRYYY TVDGQKDQTF WCDHAAIIGS NGSYNGITSN
VKGTFVKMSS STNNADTYLE ISFTGGTLEP GAHVQIQGRF
AKNDWSNYTQ SNDYSFKSAS QFVEWDQVTA YLNGVLVWGK
EP

Orthologs of type 3a CBDs have been described in various species, including, but not limited to Ruminiclostridium thermocellum AD2 (GenBank: ALX08828.1), Caldicellulosiruptor lactoaceticus 6A (GenBank: AEM74847.1), Niastella koreensis GR20-(GenBank: AEV99440.1), Actinobacteria bacterium OV450 (GenBank: KPH97519), Spirosoma linguale DSM 74 (GenBank: ADB37689.1). Type 3 CBDs, including type 3a CBDs, from other species known to one of ordinary skill in the art are also contemplated herein.

In some embodiments, the CBD includes a variant that is at least or about 50% identical, at least or about 60% identical, at least or about 70% identical, at least or about 80% identical, at least or about 85% identical, at least or about 90% identical, at least or about 95% identical, at least or about 96% identical, at least or about 97% identical, at least or about 98% identical, at least or about 99% identical, at least or about 99.5% identical, at least or about 99.9% identical, or about 100% identical to the amino acid sequence of SEQ ID NO: 1 or SEQ ID NO: 8.

In some embodiments, the type 1 CBD includes a variant that is at least or about 50% identical, at least or about 60% identical, at least or about 70% identical, at least or about 80% identical, at least or about 85% identical, at least or about 90% identical, at least or about 95% identical, at least or about 96% identical, at least or about 97% identical, at least or about 98% identical, at least or about 99% identical, at least or about 99.5% identical, at least or about 99.9% identical, or about 100% identical to the amino acid sequence of SEQ ID NO: 5.

In some embodiments, the type 3a CBD includes a variant that is at least or about 50% identical, at least or about 60% identical, at least or about 70% identical, at least or about 80% identical, at least or about 85% identical, at least or about 90% identical, at least or about 95% identical, at least or about 96% identical, at least or about 97% identical, at least or about 98% identical, at least or about 99% identical, at least or about 99.5% identical, at least or about 99.9% identical, or about 100% identical to the amino acid sequence of SEQ ID NO: 2 or SEQ ID NO: 9.

In some embodiments, the CBD includes a variant which is shorter or longer than the amino acid sequence of SEQ ID NO: 1 or SEQ ID NO: 8 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, by 200 amino acids, by 300 amino acids, by 400 amino acids, by 500 amino acids, 800 amino acids, 1000 amino acids, 1200 amino acids, 1400 amino acids or more.

In some embodiments, the type 1 CBD includes a variant which is shorter or longer than the amino acid sequence of a type 1 CBD of SEQ ID NO: 5 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more.

In some embodiments, the type 3a CBD includes a variant which is shorter or longer than the amino acid sequence of a CBD of SEQ ID NO: 2 or SEQ ID NO: 9 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more.

Any orthologs of the sequences described herein may be identified conducting a BLAST search of the sequence of interest using default parameters.

In some embodiments, the fusion protein incorporates a substrate-anchoring domain and an antibody-binding domain, in which the antibody-binding domain is expressed as a genetic fusion to the substrate-anchoring domain. In some embodiments, the antibody-binding domain is not expressed as a genetic fusion to the substrate-anchoring domain. In some embodiments, the antibody-binding domain interacts with the substrate-anchoring domain.

The fusion protein described herein can be exemplified by the use of a Nucleoprotein-CBD or a Spike-CBD fusion protein bound to a cellulose-containing substrate, such as a chromatography paper (e.g., Whatman® Grade 1 Qualitative Filtration Paper). The fusion protein bound to the cellulose-containing substrate can be contacted with a sample, such as a biological sample (e.g., blood), obtained from a subject, that contains, or is suspected to contain, an antibody that binds to a SARS-CoV-2 antigen. In some embodiments, the antibody is an antibody that binds to SARS-Cov-2 N protein; in some embodiments, the antibody is an antibody that binds to SARS-CoV-2 spike protein.

An exemplary fusion protein comprising SARS-CoV-2-Nucleoprotein-CBD comprises the sequence:

(SEQ ID NO: 6)
MGSSHHHHHHSSGLVPRGSHMSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGDGKMKDLSPRWYFYYLGTGPEAGLPYG
ANKDGIIWVATEGALNTPKDHIGTRNPANNAAIVLQLPQGTTLPKGFYAEGSRGGSQASSRSSSRSRNSSRNSTP
GSSRGTSPARMAGNGGDAALALLLLDRLNQLESKMSGKGQQQQGQTVTKKSAAEASKKPRQKRTATKAYNVTQAF
GRRGPEQTQGNFGDQELIRQGTDYKHWPQIAQFAPSASAFFGMSRIGMEVTPSGTWLTYTAAIKLDDKDPNFKDQ

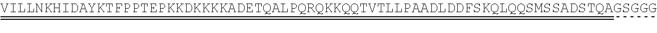

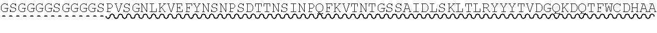

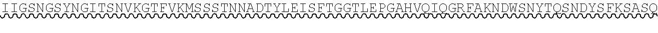

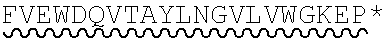

An exemplary fusion protein comprising SARS-CoV-2-Spike-CBD comprises the sequence:

(SEQ ID NO: 7)
MGSSHHHHHHSSGLVPRGSHMITNLCPFGEVFNATRFASVYAWNRKRISNCVADYSVLYNSASFSTFKC
YGVSPTKLNDLCFTNVYADSFVIRGDEVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGVGYQPYRVVVLSFELLHAPATVCGPKK

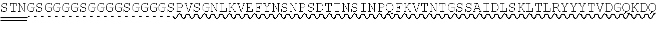

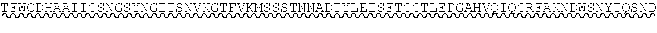

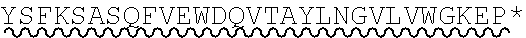

The single underlined amino acids correspond to a histidine tag-thrombin site for purification. The double underlined amino acids correspond to either SARS-CoV-2 N protein (SEQ ID NO: 6) or SARS-CoV-2 spike protein (SEQ ID NO: 7). The dash underlined amino acids correspond to the (G4S)3 linker (SEQ ID NO: 10). The zig-zag underlined amino acids correspond to the CBD. In some embodiments, any of the fusion protein constructs described herein have a similar arrangement, consisting of a purification tag and cleavage site, followed by an amino acid sequence that binds to an anti-SARS-CoV-2 N antibody, followed by a linker, and followed by the amino acid sequence of a CBD domain contemplated herein.

In some embodiments, the substrate-anchoring domain, such as a CBD, and the antibody-binding protein are directly attached. The substrate-anchoring domain, such as a CBD, can be directly attached to the antibody-binding protein through a peptide bond between the substrate-anchoring domain and the target-binding protein or antigen-binding domain. In some embodiments, the substrate-anchoring domain, such as a CBD, and the antibody-binding protein or an antibody-binding domain are indirectly attached. In some embodiments, the antibody-binding protein is indirectly attached to the CBD through a linker (i.e., is linked). Non-limiting examples of linkers contemplated herein include a protein linker; a peptide linker, such as a Gly-Ser linker (e.g., a linker that includes the amino acid sequence GGGGSGGGGSGGGGS (SEQ ID NO: 10), known as (G₄S)₃). The Gly-Ser linker can be replicated n number of times, wherein n=1, 2, 3, 4, 5, 6, 7, 8, 9, 10, 15, 20, 25, or 30, for example. Additional non-limiting examples of linkers known to one of ordinary skill in the art, such as chemical linkers (e.g., crosslinkers, bifunctional linkers, trifunctional trilinkers), such as Bis[2-(N-succinimidyl-oxycarbonyloxy)ethyl] sulfone, O,O′-Bis[2-(N-Succinimidyl-succinylamino)ethyl]polyethylene glycol 2,000, 0,0′-Bis[2-(N-Succinimidyl-succinylamino)ethyl]polyethylene glycol 3,000, 0,0′-Bis[2-(N-Succinimidyl-succinylamino)ethyl]polyethylene glycol 10,000, BS(PEG)5 (PEGylated bis(sulfosuccinimidyl)suberate), 4,4′-Diisothiocyanatostilbene-2,2′-disulfonic acid disodium salt hydrate, bromoacetic acid N-hydroxysuccinimide ester, maleimide-PEG2-succinimidyl ester, SBAP (succinimidyl 3-(bromoacetamido)propionate), 5-Azido-2-nitrobenzoic acid N-hydroxysuccinimide ester, etc.; flexible linkers (e.g., (Gly)₆ (SEQ ID NO: 11), (Gly)s (SEQ ID NO: 12), etc.), rigid linkers (e.g., (EAAAK)₃ (SEQ ID NO: 13), A(EAAAK)₄ALEA(EAAAK)₄A (SEQ ID NO: 14), PAPAP (SEQ ID NO: 15), etc.) and cleavable linkers (e.g., disulfide, VSQTSKLTR↓JAETVFPDV (SEQ ID NO: 16), RVL↓AEA (SEQ ID NO: 17); EDVVCC↓SMSY (SEQ ID NO: 18); GGIEGR↓GS (SEQ ID NO: 19); GFLG↓ (SEQ ID NO: 20), etc.) naturally-occurring or synthetic, such as those disclosed in Chen et al., Adv Drug Deliv Rev (2013) 65(10):1357-69, are also contemplated herein.

In some embodiments, the C-terminus of the antibody-binding protein is either directly or indirectly attached to the N-terminus of the CBD. In some embodiments, the C-terminus of the antibody-binding protein is directly attached to the N-terminus of the CBD. In some embodiments, the C-terminus of the antibody-binding protein is indirectly attached to the N-terminus of the CBD through a linker. In some embodiments, the N-terminus of the antibody-binding protein is either directly or indirectly attached to the C-terminus of the CBD. In some embodiments, the N-terminus of the antibody-binding protein is directly attached to the C-terminus of the CBD. In some embodiments, the N-terminus of the antibody-binding protein is indirectly attached to the C-terminus of the CBD through a linker.

In some embodiments, the fusion protein comprises more than one antibody-binding domain. In some embodiments, the fusion protein comprises at least or 2, at least or 3, at least or 4, at least or 5, at least or 6, at least or 7, at least or 8, at least or 9, at least or 10, at least or 12, at least or 14, at least or 16, at least or 18, at least or 20, at least or 25, at least or 30, at least or 35, at least or 40, at least or 45, at least or 50, at least or 55, at least or 60, at least or 65, at least or 70, at least or 75, at least or 80, at least or 85, at least or 90, at least or 95, or at least or 100 antibody-binding proteins or domains.

In some embodiments, the more than one antibody-binding proteins or domains, such as any of the variants disclosed herein, are genetically fused together. The more than one antibody-binding proteins or domains, such as any of the variants disclosed herein, are genetically fused together by using an expression vector that includes more than one copy of a nucleic acid sequence that encodes the antibody-binding protein or domain. In some embodiments, the nucleic acid sequences that encodes one antibody-binding protein or domain is separated from another nucleic acid sequence that encodes one antigen-binding protein or domain by a nucleic acid encoding a linker. In some embodiments, the more than one antibody-binding proteins or domains, such as any of the variants disclosed herein are not genetically fused together. In some embodiments, the more than one antibody-binding proteins or domains, such as any of the variants disclosed herein are chemically fused. The more than one antibody-binding proteins or domains, such as any of the variants disclosed herein, are chemically fused by a chemical reagent after the proteins have been expressed from a nucleic acid sequence. In some embodiments, the more than one antibody-binding proteins or domains, such as any of the variants disclosed herein, are chemically fused after antigen-binding proteins or domains, such as any of the variants disclosed herein, is expressed, for instance, from an expression vector. In some embodiments, the more than one antibody-binding proteins are chemically fused by a linker, such as a bifunctional linker, or using other methods known to one of ordinary skill in the art. In some embodiments, the more than one antibody-binding proteins or domains, such as any of the variants disclosed herein, are chemically fused by a fusion via disulfide linkages between cysteine residues at the N- and C-termini, or via dual-maleimide chemical reagents. In some embodiments, in vivo ligation tags such as HALO or SPY tags to attach orthogonal reactive moieties to the antibody-binding proteins or domains, such as any of the variants disclosed herein, allowing separate molecules to react together, are also contemplated herein. In some embodiments, residues of antibody-binding proteins or domains, such as any of the variants disclosed herein, could be chemically altered to feature aldehyde moieties, which can be reacted with primary amines to form covalent imine linkages. (See e.g., Tuley et al., Chemical communications (2014) 50(56):7424-7426. doi:10.1039/c4cc02000f). In some embodiments, a sortase-based method could be used for in vitro fusion of an antibody-binding protein or domain, such as any of the variants disclosed herein.

### Expression of Fusion Protein

Also disclosed herein are nucleic acids that encode for any of the fusion proteins described herein, libraries that contain any of the nucleic acids and/or fusion proteins described herein, and compositions that contain any of the nucleic acids and/or fusion proteins described herein. It should be appreciated that libraries containing nucleic acids or proteins can be generated using methods known in the art. A library containing nucleic acids can contain fragments of genes and/or full-length genes and can contain wild-type sequences and mutated sequences. A library containing proteins can contain fragments of proteins and/or full length proteins and can contain wild-type sequences and mutated sequences.

In some embodiments, one or more of the antibody-binding proteins disclosed herein are expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA, although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA).

A nucleic acid molecule that encodes a fusion protein or antigen or any other molecule disclosed herein can be introduced into a cell or cells using methods and techniques that are standard in the art. For example, nucleic acid molecules can be introduced by standard protocols such as transformation including chemical transformation and electroporation, transduction, particle bombardment, etc.

Any type of cell that can be engineered to recombinantly express genes can be used in the methods described herein, including prokaryotic and eukaryotic cells. In some embodiments the cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus. In other embodiments, the cell is a fungal cell such as a yeast cell, e.g., Saccharomyces spp. (e.g., S. cerevisiae), Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp. and industrial polyploid yeast strains. Other examples of fungi include Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In other embodiments, the cell is an algal cell, or a plant cell.

### Compositions

In some aspects, compositions of the fusion proteins described herein are also provided. In some embodiments, the composition includes any of the fusion proteins described herein bound to a cellulose-containing substrate. In some embodiments, the cellulose-containing substrate is paper (e.g., chromatography paper) or nitrocellulose. In certain embodiments, the cellulose-containing substrate is modified in an oxidizing chemical bath to yield covalent chemical linkage of the protein to the substrate, passivated with a blocking agent to reduce non-specific protein adsorption to the substrate, or pre-incubated with a stabilizing species such as trehalose in order to improve assay functionality and stability (See e.g., Y. Zhu, et al., Anal Chem. (2014) 86:2871-5; M. Vuoriluoto, et al., ACS Appl. Mater. Interfaces (2016) 8, 5668-78). In certain embodiments, the cellulose-containing substrate is not modified (unmodified). In some embodiments, the cellulose-containing substrate is an unmodified chromatography paper, such as unmodified Whatman® Grade 1 Qualitative Filtration Paper. Additional non-limiting examples of cellulose-containing substrates also contemplated herein include cellulose powder, cellulose microbeads, or cellulosic fabrics/yams.

In some embodiments, the cellulose-containing substrate is oxidized. In some embodiments, the cellulose-containing substrate is oxidized with sodium metaperiodate to functionalize the cellulose surfaces with aldehyde groups or other methods to oxidize cellulose known to one of ordinary skill in the art. (See e.g., Badu-Tawiah, et al., Lab Chip, (2015) 15:655-9).

In some embodiments, at least or about 0.1 micromole, at least or about 0.2 micromoles, at least or about 0.3 micromoles, at least or about 0.4 micromoles, at least or about 0.5 micromoles, at least or about 0.6 micromoles, at least or about 0.7 micromoles, at least or about 0.8 micromoles, at least or about 0.9 micromoles, at least or about 1 micromole, at least or about 1.1 micromoles, at least or about 1.2 micromoles, at least or about 1.3 micromoles, at least or about 1.4 micromoles, at least or about 1.5 micromoles, at least or about 1.6 micromoles, at least or about 1.7 micromoles, at least or about 1.8 micromoles, at least or about 1.9 micromoles, at least or about 2 micromoles, at least or about 2.1 micromoles, at least or about 2.2 micromoles, at least or about 2.3 micromoles, at least or about 2.4 micromoles, at least or about 2.5 micromoles, at least or about 2.6 micromoles, at least or about 2.7 micromoles, at least or about 2.8 micromoles, at least or about 2.9 micromoles, at least or about 3 micromoles, at least or about 3.5 micromoles, at least or about 4 micromoles, at least or about 4.5 micromoles, or at least or about 5 micromoles of any of the fusion proteins described herein are attached to a cellulose-containing substrate per gram of cellulose of the cellulose-containing substrate.

In some embodiments, at least or about 1 μM, at least or about 25 μM, at least or about 50 μM, at least or about 60 μM, at least or about 70 μM, at least or about 80 μM, at least or about 90 μM, at least or about 100 μM, at least or about 150 μM, at least or about 200 M, at least or about 250 μM, at least or about 300 μM, at least or about 350 μM, at least or about 400 μM, at least or about 500 μM, at least or about 550 μM, at least or about 600 μM, at least or about 650 μM, at least or about 700 μM, at least or about 750 μM, at least or about 800 μM, at least or about 850 μM, at least or about 900 μM, at least or about 950 μM, at least or about 1 mM, at least or about 1.5 mM, at least or about 2 mM, at least or about 2.5 mM, at least or about 3 mM, at least or about 3.5 mM, at least or about 4 mM, at least or about 4.5 mM, at least or about 5 mM of volume-average concentration of any of the fusion proteins described herein are attached to a cellulose-containing substrate.

### Antibody Detection Methods

In some aspects, methods for detecting an anti-SARS-CoV-2 antibody are also provided herein. In some embodiments, the method includes contacting any of the fusion proteins described herein with a cellulose-containing substrate for a time sufficient for the fusion protein to bind to the cellulose-containing substrate; contacting the fusion protein bound to the cellulose-containing substrate with a sample comprising, or suspected to comprise, an anti-SARS-CoV-2 antibody; and detecting the antibody, if present, bound to the antibody-binding protein.

In some embodiments, the method includes contacting any of the fusion proteins described herein with a sample comprising, or suspected to comprise, an anti-SARS-CoV-2 antibody, wherein the anti-SARS-CoV-2 antibody binds to the fusion protein and forms a complex; contacting the complex with a cellulose-containing substrate for a time sufficient for the complex to bind to the cellulose-containing substrate; and detecting the antibody, if present, bound to the antibody-binding protein.

In some embodiments, the method includes contacting any of the fusion proteins described herein with a cellulose-containing substrate for a time sufficient for fusion protein to bind to the cellulose-containing substrate; contacting a sample, such as a biological sample, comprising, or suspected to comprise, an anti-SARS-CoV-2 antibody for a time sufficient to allow the anti-SARS-CoV-2 antibody to bind to the fusion protein and form a complex.

In an aspect, the method further includes detecting the antibody, if present. In some embodiments, detecting comprises addition of a second, detectably-labeled antibody that binds to the anti-SARS-CoV-2 antibody. The detectably-labeled antibody comprises a detectable label, which may be a signal-generating reagent as described elsewhere herein. In some embodiments, the detectably-labeled antibody is an enzyme-labeled antibody. In some embodiments, the enzyme-labeled antibody binds to a human antibody. In some embodiments, the enzyme-labeled antibody binds to a human IgG or human IgM antibody. In some embodiments, the enzyme-labeled antibody is labeled with horseradish peroxidase (HRP).

In some embodiments, the method comprises contacting the complex described above with a second antibody that recognizes the anti-SARS-CoV-2 antibody; and detecting the second antibody. In some embodiments, the second antibody is directly or indirectly linked to a fluorophore or a molecule that emits a detectable signal to detect the anti-SARS-CoV-2 antibody. In some embodiments, the second antibody is biotinylated. In some embodiments, the biotinylated antibody is contacted with a streptavidin molecule that is directly or indirectly linked to a fluorophore or a molecule that emits a detectable signal to detect the antigen or antigen of interest.

In some embodiments, the method includes contacting any of the fusion proteins described herein with a sample comprising, or suspected to comprise, an anti-SARS-CoV-2 antibody, wherein the anti-SARS-CoV-2 antibody binds to the fusion protein and forms a first complex; contacting the first complex with a second antibody for detection, as described above, wherein the first complex and the second antibody form a second complex; contacting the second complex with a cellulose-containing substrate for a time sufficient for the complex to bind to the cellulose-containing substrate; and detecting the antibody, if present, bound to the antibody-binding protein.

In some embodiments, the fusion protein or the complex is in solution. In some embodiments, the solution includes a buffer, such as a buffer known to one of ordinary skill in the art. The bifunctional protein may be in solution at a desired concentration. In some embodiments, the fusion protein is at a desired concentration of or about 5 μM, of or about 10 M, of or about 15 μM, of or about 20 μM, of or about 25 μM, of or about 30 μM, of or about M, of or about 40 μM, of or about 45 μM, of or about 50 μM, of or about 60 μM, of or about 70 μM, of or about 80 μM, of or about 90 μM, of or about 100 μM, of or about 200 M, of or about 300 μM, or of or about 400 μM.

In some embodiments, the fusion protein described herein is contacted with the cellulose-containing substrate for about 5 seconds, about 15 seconds, about 20 seconds, about 30 seconds, about 35 seconds, about 40 seconds, about 45 seconds, about 1 minute, about 1.5 minutes, about 2 minutes, about 2.5 minutes, about 3 minutes, about 4 minutes, about 5 minutes, about 7 minutes, about 10 minutes, about 15 minutes, about 20 minutes, about 30 minutes, or about 1 hour.

In some embodiments, the sample is a biological sample. The biological sample may be obtained from a subject. As described herein, the term “biological sample” is used to generally refer to any biological material obtained from a subject. The biological sample typically is a fluid sample. Solid tissues may be made into fluid samples using routine methods in the art. In some embodiments, the biological sample is tissue, feces, or a cell obtained from a subject. In some embodiments, the biological sample comprises a bodily fluid from a subject. The bodily fluids can be fluids isolated from anywhere in the body of the subject, preferably a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, intra-organ system fluid, ascitic fluid, tumor cyst fluid, amniotic fluid or combinations thereof.

For instance, a non-limiting example is the use of SARS-CoV-2 antigen-CBD fusion protein bound to a cellulose-containing substrate, such as a chromatography paper (e.g., Whatman® Grade 1 Qualitative Filtration Paper), which is contacted with a sample that contains, or is suspected to contain, an anti-SARS-CoV-2 antibody, such as an blood sample obtained from a subject that has been, or is suspected of having been, exposed to SARS-CoV-2. In some instances, the result may be used to determine whether the subject has been exposed to SARS-CoV-2. In some embodiments, the result may be used to decide whether the subject is susceptible to SARS-CoV-2 reinfection.

In some aspects, the molar abundance or molar excess of the antibody-binding protein in the fusion protein, such as a SARS-CoV-2 antigen linked to a CBD, relative to the anti-SARS-CoV-2 antibody of interest allows the rapid capture and, in some embodiments, efficient and complete depletion of the antibody of interest from a sample.

In some embodiments, at least or about a 10-fold molar excess of fusion protein or antibody-binding protein completely depletes an anti-SARS-CoV-2 antibody of interest from a sample or solution. In some embodiments, at least or about a 10-fold volume-average concentration excess leads to rapid capture and/or immobilization of a fusion protein or antibody-binding protein.

In some embodiments, the fusion protein is in molar excess of the anti-SARS-CoV-2 antibody of interest. In some embodiments, the fusion protein is in at least or about 2-fold molar excess, at least or about 3-fold molar excess, at least or about 4-molar excess, at least or about 5-fold molar excess, at least or about 6-fold molar excess, at least or about 7-fold molar excess, at least or about 8-fold molar excess, at least or about 9-fold molar excess, at least or about 10-fold molar excess, at least or about 15-fold molar excess, at least or about 20-fold molar excess, at least or about 25-fold molar excess, at least or about 30-fold molar excess, at least or about 35-fold molar excess, at least or about 40-fold molar excess, at least or about 45-fold molar excess, at least or about 50-fold molar excess, at least or about 60-fold molar excess, at least or about 65-fold molar excess, at least or about 70-fold molar excess, at least or about 80-fold molar excess, at least or about 90-fold molar excess, at least or about 100-fold molar excess, at least or about 200-fold molar excess, at least or about 300-fold molar excess, at least or about 400-fold molar excess, at least or about 500-fold molar excess, at least or about 600-fold molar excess, at least or about 700-fold molar excess, at least or about 800-fold molar excess, at least or about 900-fold molar excess, at least or about 1000-fold molar excess, at least or about 1500-fold molar excess, or at least or about 2000-fold molar excess relative to the anti-SARS-CoV-2 antibody of interest in the sample.

In some embodiments, the fusion protein is in such excess that the antigen of interest is depleted from the sample. In some embodiments, about or at least 10%, about or at least 20%, about or at least 30%, about or at least 40%, about or at least 50%, about or at least 55%, about or at least 60%, about or at least 65%, about or at least 70%, about or at least 75%, about or at least 80%, about or at least 81%, about or at least 82%, about or at least 83%, about or at least 84%, about or at least 85%, about or at least 86%, about or at least 87%, about or at least 88%, about or at least 89%, about or at least 90%, about or at least 91%, about or at least 92%, about or at least 93%, about or at least 94%, about or at least 95%, about or at least 95.5%, about or at least 96%, about or at least 96.5%, about or at least 97%, about or at least 97.5%, about or at least 98%, about or at least 98.5%, about or at least 99%, about or at least 99.5%, or about 100% of the antigen of interest is depleted from the sample, such as a biological sample.

In some aspects, standard curves can be prepared given the advantageous properties of the disclosure in which complete or near-complete depletion of an anti-SARS-CoV-2 antibody can be achieved from a sample or solution. The abundance of the captured anti-SARS-CoV-2 antibody can be detected and measured or determined using a readout, such as a fluorescent readout or a colorimetric readout.

In some embodiments, the surface-immobilized concentration of the antibody-binding protein (e.g., nucleoprotein-CBD) is quantified using a protein assay, such as a micro bicinchoninic acid (BCA) assay. A standard curve can be prepared by evaporating known quantities of protein onto cellulose test zones, depositing these test zones into the wells of a micro BCA assay, and quantifying the signal development in this format. The same procedure is followed for the experimental samples (following the substrate washing step), and the associated signal for each sample is then mapped to this standard curve in order to determine the mass of immobilized nucleoprotein-CBD or spike-CBD.

In some embodiments, the sample is a biological sample from a subject. A subject includes, but is not limited to, any mammal, such as a human, a primate, a mouse, a rat, a dog, a cat, a horse, or agricultural stocks (e.g., fish, pigs, cows, sheep, and birds—particularly chickens). In certain embodiments, the subject is a human. In some embodiments, the sample is a solution, such as a buffer solution.

In some embodiments, the cellulose-containing substrate is rinsed with a buffer solution before detecting the anti-SARS-CoV-2 antibody bound to the engineered antibody-binding protein. In some embodiments, the buffer is phosphate buffered saline (PBS) or another buffer known to one of ordinary skill in the art that provides a stable environment for a macromolecule, such as a protein, protein complex, antigen, etc.

In some embodiments, the method further includes detecting the anti-SARS-CoV-2 antibody of interest bound by the engineered antibody-binding protein (e.g., SARS-CoV-2 nucleoprotein or spike protein) in the fusion protein. In some embodiments, the anti-SARS-CoV-2 antibody bound to the fusion protein is contacted with a cellulose-containing substrate in which the CBD of the fusion protein binds the cellulose-containing substrate (e.g., chromatography paper such as Whatman® Grade 1 Qualitative Filtration Paper). The method allows for the separation or isolation of the anti-SARS-CoV-2 antibody from any other molecules that may be present in a sample, such as a biological sample (e.g., blood). In some embodiments, the presence or amount of the anti-SARS-CoV-2 antibody is determined or measured using a signal-generating reagent that specifically recognizes the anti-SARS-CoV-2 antibody and generates a signal.

In some embodiments, the fusion protein (e.g., Nucleoprotein-CBD) is immobilized on a cellulose substrate (e.g., chromatography paper, cellulose powder, etc.), and then is brought into contact with the solution/biological sample bearing the anti-SARS-CoV-2 antibody (either forced convection to draw the fluid across or through the test zone, or co-incubation of the CBD/substrate and antibody). This immobilized complex would then be contacted with a detection moiety, such as an epitope-specific binding molecule (optionally fused to a biotin acceptor sequence, or modified with a fluorophore). The detection moiety (e.g., an antibody that binds the anti-SARS-CoV-2 antibody, such as a constant region of the antibody) binds to an epitope of the captured antibody. This detection moiety is conjugated to a means of transducing this binding reaction, such as a signal-generating reagent or a detection reagent; several examples are outlined below. All of these steps can be done directly on the cellulose-containing substrate.

Non-limiting examples of signal-generating reagent that can be fused to the detection moiety include, without limitation, gold nanoparticles, enzymes (expressed as fusion partners or indirectly bound to the detection moiety) which yield a colorimetric response, enzymes which yield an amperometric or impedometric signal (e.g., glucose oxidase), a macrophotoinitiator which can initiate a polymerization reaction, cellulose nanobeads, other metallic nanoparticles, dye-filled liposomes, DNA which can be amplified enzymatically, RNA which can be expressed for the production of a color-producing enzyme, etc. The presence or amount of the signal-generating reagent can be detected using an imaging device, such as a digital imager. Additional non-limiting examples of detecting the signal-generating reagent include gold nanoparticles, which can be used in a point-of-care setting, and are the reagents used in traditional pregnancy tests. The spatial localization of gold nanoparticles, mediated by the antigen-binding interaction, concentrates the optical signal (which is also amplified by the occurrence of surface plasmon resonance). This can be detected by the naked eye. Polymerization-based amplification would use the localization of a macrophotoinitiator in order to yield a rapid, durable polymerization response following incubation with a monomer solution and irradiation with the appropriate wavelength of light. Entrained phenolphthalein yields a high-contrast colorimetric readout following the application of a basic solution, which can be detected with the naked eye. An amperometric method, such as fusing glucose oxidase to a detection species and contacting the tests with gold probes and a glucose solution, would allow for smart phone based detection. Enzymatic methods can also be used, and rely upon a fusion of the second species (e.g., rcSso7d) to an enzyme and contacting the tests with a labile substrate which becomes colored following enzymatic cleavage. Impedometric means of detecting the signal generating reagent are also possible, and can be achieved using smartphone-compatible adaptors.

In some embodiments, the detection reagent is a fluorophore. In some embodiments, the fluorophore is hydroxycoumarin, methoxycoumarin, aminocoumarin, Cy2, FAM, Alexa Fluor 647 (AF647), Alexa Fluor 405 (AF405), Alexa Fluor 488 (AF488), Fluorescein FITC, Alexa Fluor 430 (AF430), Alexa Fluor 532 (AF532), HEX, Cy3, TRITC, Alexa Fluor 546 (AF546), Alexa Fluor 555 (AF555), R-phycoerythrin (PE), Rhodamine Red-X, Tamara, Cy3.5 581, Rox, Alexa Fluor 568 (AF568), Red 613, Texas Red, Alexa Fluor 594 (AF594), Alexa Fluor 633 (AF633), Allophycocyanin, Cy5, Alexa Fluor 660 (AF660), Cy5.5, TruRed, Alexa Fluor 680 (AF680), Cy7, Cy7.5 or any other fluorophores known to one of ordinary skill in the art (see e.g., www.biosyn.com/Images/Articlelmages/Comprehensive %20fluorophore %20list.pdf). In some embodiments, the fluorophore is a fluorescent protein or a chromophore, such as green fluorescent protein (GFP), chromoprotein from the coral Acropora millepora (amilCP), a chromoprotein from the coral Acropora millepora (amilGFP), a fluorescent protein from Acropora millepora (amilRFP), etc., or other species chemically linked to a detection reagent known to one of ordinary skill in the art. In some embodiments, one or more fluorophores could be used for the purification of chemically-labeled molecules to ensure 100% or near 100% labeling efficiency.

In some embodiments, the detection reagent is a molecule that emit a detectable signal. In some embodiments, the molecule is phycoerythrin. In some embodiments, the molecule that emits a detectable signal is a color-producing enzyme (e.g., beta-galactosidase), APEX2 for metal sequestration and high contrast electron microscopy (EM), or a chemiluminescent species. Other detection reagents, fluorophores or molecules that emit a detectable signal known to one of ordinary skill in the art are also contemplated herein. In some embodiments, the detection reagent, fluorophore or molecule that emits a detectable signal is directly or indirectly linked to one or more of streptavidin, to IgG antibody (polyclonal or monoclonal), any of the biomarkers disclosed herein, any of the antibody-binding proteins disclosed herein, a nucleic acid (e.g., DNA, RNA, etc.), or an organic or inorganic nanoparticle (e.g., a nanoparticle comprising gold, carbon, latex, cellulose, etc.)

### Kits

In some aspects, the fusion protein and compositions described herein are provided in a kit. In some embodiments, the kit is used to assess the presence or amount of a molecule, such as an anti-SARS-CoV-2 antibody of interest and includes a container containing any of the fusion proteins described herein.

In some embodiments, the kit further comprises a cellulose-containing substrate. In some embodiments, the fusion protein is bound to the cellulose-containing substrate, as disclosed above.

### EXAMPLES

### Example 1. Paper-Based Diagnostics for Detecting Antibodies to SARS-CoV-2 N Protein and Spike Protein

Test Proteins were Produced Using E. coli.

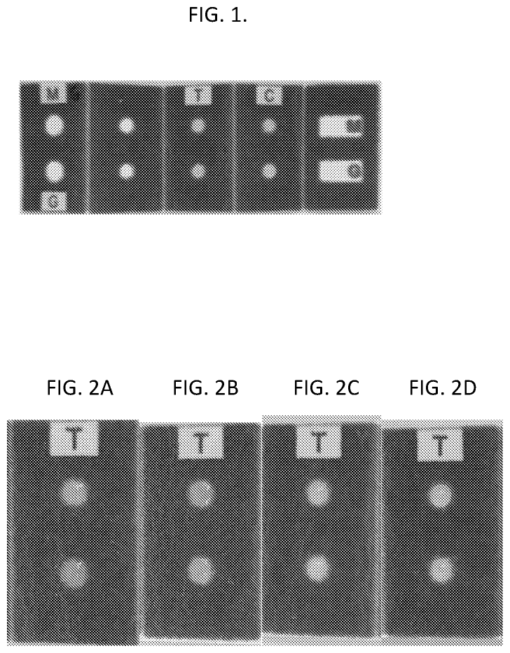

Blood plasma samples that were confirmed positive or confirmed negative for SARS-CoV-2 antibodies using established laboratory techniques (RNA tests at the time of diagnosis, ELISA tests after recovery) were analyzed using paper devices as shown in FIG. 1.

The test zones were modified with engineered SARS-CoV-2 nucleoprotein-CBD. The top spot in the T column tests for IgM, and the bottom spot in the T column tests for IgG. The C column is for controls.

### Methods

Patient samples were tested by the following method. 4 μL of undiluted plasma sample was added to 50 μL of detection reagent. The detection reagents used were: 0.2 nM-0.05 nM HRP-conjugated anti-human IgM or 0.2 nM HRP-conjugated anti-human IgG antibody.

Each prepared sample (using either IgG or IgM detection reagents) was applied to the test wells of the device, labeled M and G in FIG. 1. 50 μL of 1×PBS was then added to promote flow through of unbound detection reagents.

After allowing time for all added solutions to be absorbed, the device was unfolded and 5 μL of TMB/H2O2 solution was applied to test and control wells. Results are shown in FIG. 2A, FIG. 2B, FIG. 2C, and FIG. 2D.

### REFERENCES

Ackerman, M., Levary, D., Tobon, G., Hackel, B., Orcutt, K. D., Wittrup, K. D., 2009. Biotechnol. Prog. 25, 774-783.
Chao, G., Lau, W. L., Hackel, B. J., Sazinsky, S. L., Lippow, S. M., Wittrup, K. D., 2006. Nat. Protoc. 1, 755-68.
Miller, E. A., Traxlmayr, M. W., Shen, J., Sikes, H. D., 2016. Mol. Syst. Des. Eng. 1, 377-381.
Tomme, P., Boraston, A., McLean, B., Kormos, J., Creagh, A. L., Sturch, K., Gilkes, N. R., Haynes, C. A., Warren, R. A. J., Kilburn, D. G., 1998. J. Chromatogr. B Biomed. Appl. 715, 283-296.
Traxlmayr, M. W., Kiefer, J. D., Srinivas, R. R., Lobner, E., Tisdale, A. W., Mehta, N. K., Yang, N.J., Tidor, B., Wittrup, K. D., 2016. J. Biol. Chem. 291, 22496-22508.
Vuoriluoto, M., Orelma, H., Zhu, B., Johansson, L.-S. S., Rojas, O. J., 2016. ACS Appl. Mater. Interfaces 8, 5668-5678.
Zhu, Y., Xu, X., Brault, N. D., Keefe, A. J., Han, X., Deng, Y., Xu, J., Yu, Q., Jiang, S., 2014. Anal. Chem. 86, 2871-2875.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A fusion protein comprising a cellulose binding domain (CBD) and a protein which binds an antibody to SARS-CoV-2.

2. The fusion protein of claim 1, wherein the C-terminus of the antibody-binding protein is linked to the N-terminus of the CBD.

3. The fusion protein of claims 1 or 2, wherein the antibody-binding protein is linked to the CBD through a linker.

4. The fusion protein of claim 3, wherein the linker is a Gly-Ser linker.

5. The fusion protein of any one of claims 1-4, wherein the antibody-binding protein comprises a SARS-CoV-2 protein.

6. The fusion protein of any one of claims 1-5, wherein the antibody-binding protein comprises a SARS-CoV-2 nucleoprotein or a fragment thereof.

7. The fusion protein of any one of claims 1-5, wherein the antibody-binding protein comprises a SARS-CoV-2 spike protein or a fragment thereof.

8. The fusion protein of any one of claims 1-6, wherein the antibody-binding protein comprises at least 85% sequence identity to the amino acid sequence of SEQ ID NO: 3.

9. The fusion protein of any one of claims 1-6, wherein the antibody-binding protein comprises at least 90% sequence identity to the amino acid sequence of SEQ ID NO: 3.

10. The fusion protein of any one of claims 1-6, wherein the antibody-binding protein comprises at least 95% sequence identity to the amino acid sequence of SEQ ID NO: 3.

11. The fusion protein of any one of claims 1-6, wherein the antibody-binding protein comprises at least 99% sequence identity to the amino acid sequence of SEQ ID NO: 3.

12. The fusion protein of any one of claims 1-6, wherein the antibody-binding protein sequence is 100% identical to the amino acid sequence of SEQ ID NO: 3.

13. The fusion protein of any one of claims 1-5 and 7, wherein the antibody-binding protein comprises at least 85% sequence identity of the amino acid sequence of SEQ ID NO: 4.

14. The fusion protein of any one of claims 1-5 and 7, wherein the antibody-binding protein comprises at least 90% sequence identity to the amino acid sequence of SEQ ID NO: 4.

15. The fusion protein of any one of claims 1-5 and 7, wherein the antibody-binding protein comprises at least 95% sequence identity to the amino acid sequence of SEQ ID NO: 4.

16. The fusion protein of any one of claims 1-5 and 7, wherein the antibody-binding protein comprises at least 99% sequence identity to the amino acid sequence of SEQ ID NO: 4.

17. The fusion protein of any one of claims 1-5 and 7, wherein the antibody-binding protein sequence is 100% identical to the amino acid sequence of SEQ ID NO: 4.

18. The fusion protein of any one of claims 1-17, wherein the CBD is a type 3a CBD, or the type 1 dimerized CBD (dCBD).

19. The fusion protein of claim 18, wherein the type 3a CBD is a domain of the protein CipA from Clostridium thermocellum.

20. The fusion protein of any one of claims 1-19, wherein the fusion protein sequence is SEQ ID NO: 13.

21. The fusion protein of any one of claims 1-19, wherein the fusion protein sequence is SEQ ID NO: 14.

22. A method for detecting an anti-SARS-CoV-2 antibody, the method comprising:
(a) contacting the fusion protein of any one of claims 1-21 with a cellulose-containing substrate for a time sufficient for the fusion protein to bind the cellulose-containing substrate;
(b) contacting the fusion protein bound to the cellulose-containing substrate with a sample comprising or suspected to comprise an anti-SARS-CoV-2 antibody; and
(c) detecting the anti-SARS-CoV-antibody, if present, bound by the antibody-binding protein.

23. The method of claim 22, wherein detecting comprises addition of a detectably-labeled antibody that binds to the anti-SARS-CoV-2 antibody.

24. The method of claim 23, wherein the detectably-labeled antibody is an enzyme-labeled antibody

25. The method of claim 24, wherein the enzyme-labeled antibody binds to a human antibody.

26. The method of claim 25, wherein the enzyme-labeled antibody binds to a human IgG or human IgM antibody.

27. The method of any one of claims 24-26, wherein the enzyme-labeled antibody is labeled with horseradish peroxidase (HRP).

28. The method of any one of claims 22-27, wherein the fusion protein is in molar excess of the anti-SARS-CoV-2 antibody.

29. The method of claim 28, wherein the fusion protein is in at least 10-fold molar excess of the anti-SARS-CoV-2 antibody.

30. The method of any one of claims 22-29, wherein the anti-SARS-CoV-2 antibody is an anti-SARS-CoV-2 antibody that binds to SARS-CoV-2 nucleoprotein or SARS-CoV-2 spike protein.

31. The method of any one of claims 22-30, wherein at least 50% of the anti-SARS-CoV-2 antibody is bound by the fusion protein.

32. The method of any one of claims 22-31, wherein the cellulose-containing substrate is paper, nitrocellulose, or cellulose powder.

33. The method of claim 32 wherein the paper is chromatography paper.

34. The method of claim 33, wherein the chromatography paper is unmodified.

35. The method of any one of claims 22-34, further comprising rinsing the cellulose-containing substrate with a buffer solution before detecting the anti-SARS-CoV-2 antibody bound by the antibody-binding protein.

36. The method of any one of claims 22-35, wherein the sample is a biological sample from a subject.

37. The method of claim 36, wherein the subject is a mammal.

38. The method of claim 36, wherein the subject is a human.

39. The method of any one of claims 36-38, further comprising providing treatment to the subject.

40. A method for detecting an anti-SARS-CoV-2 antibody of interest, the method comprising:
(a) contacting the fusion protein of any one of claims 1-21 with a sample comprising or suspected to comprise an anti-SARS-CoV-2 antibody, wherein the anti-SARS-CoV-2 antibody binds to the fusion protein and forms a complex;
(b) contacting the complex with a cellulose-containing substrate for a time sufficient for the complex to bind to the cellulose-containing substrate; and
(c) detecting the anti-SARS-CoV-2 antibody, if present, bound by the antibody-binding protein.

41. The method of claim 40, wherein detecting comprises addition of a detectably-labeled antibody that binds to the anti-SARS-CoV-2 antibody.

42. The method of claim 41, wherein the detectably-labeled enzyme is an enzyme-labeled antibody.

43. The method of claim 42, wherein the enzyme-labeled antibody binds to a human antibody.

44. The method of claim 43, wherein the enzyme-labeled antibody binds to a human IgG or human IgM antibody.

45. The method of any one of claims 42-44, wherein the enzyme-labeled antibody is labeled with horseradish peroxidase (HRP).

46. The method of any one of claims 40-45, wherein the fusion protein is in molar excess of the anti-SARS-CoV-2 antibody.

47. The method of claim 46, wherein the fusion protein is in at least 10-fold molar excess of the anti-SARS-CoV-2 antibody.

48. The method of any one of claims 40-47, wherein at least 50% of the anti-SARS-CoV-2 antibody is depleted from the sample.

49. The method of any one of claims 40-48, wherein the cellulose-containing substrate is paper, nitrocellulose, or cellulose powder.

50. The method of claim 49, wherein the paper is chromatography paper.

51. The method of claim 50, wherein the chromatography paper is unmodified.

52. The method of any one of claims 40-51, further comprising rinsing the cellulose-containing substrate with a buffer solution before detecting the anti-SARS-CoV-2 antibody bound by the antibody-binding protein.

53. The method of any one of claims 40-52, wherein the sample is a biological sample from a subject.

54. The method of claim 53, wherein the subject is a mammal.

55. The method of claim 54, wherein the subject is a human.

56. The method of any one of claims 53-55, further comprising providing treatment to the subject.

57. A method for assessing a presence or amount of an anti-SARS-CoV-2 antibody in a sample, the method comprising contacting the sample with the fusion protein of any one of claims 1-21 and measuring the presence or amount of the anti-SARS-CoV-2 antibody in the sample.

58. The method of claim 57, wherein the fusion protein is in molar excess of the anti-SARS-CoV-2 antibody.

59. The method of claim 58, wherein the fusion protein is in at least 10-fold molar excess of the anti-SARS-CoV-2 antibody.

60. The method of any one of claims 57-59, wherein at least 50% of the anti-SARS-CoV-2 antibody is bound to the fusion protein.

61. The method of any one of claims 57-60, wherein the fusion protein is bound to a cellulose-containing substrate.

62. The method of claim 61, wherein the cellulose-containing substrate is paper, nitrocellulose, or cellulose powder.

63. The method of claim 62, wherein the paper is chromatography paper.

64. The method of claim 63, wherein the chromatography paper is unmodified.

65. The method of any one of claims 57-64, wherein the sample is a biological sample from a subject.

66. The method of claim 65, wherein the subject is a mammal.

67. The method of claim 66, wherein the subject is a human.

68. The method of any one of claims 65-67, further comprising providing treatment to the subject.

69. The method of any one of claims 57-68, wherein the CBD is bound to the cellulose-containing substrate.

70. A composition comprising the fusion protein of any one of claims 1-21 bound to a cellulose-containing substrate.

71. The composition of claim 70, wherein the cellulose-containing substrate is paper, nitrocellulose, or cellulose powder.

72. The composition of claim 71, wherein the paper is chromatography paper.

73. The composition of claim 72, wherein the chromatography paper is unmodified.

74. A kit for assessing a presence or amount of an anti-SARS-CoV-2 antibody, the kit comprising a container containing the fusion protein of any one of claims 1-21.

75. The kit of claim 74, further comprising a cellulose-containing substrate.

76. The kit of claim 75, wherein the fusion protein is bound to the cellulose-containing substrate.

77. The kit of claim 75, wherein the fusion protein is not bound to the cellulose-containing substrate.

78. The kit of any one of claims 74-77, wherein the cellulose-containing substrate is paper, nitrocellulose, or cellulose powder.

79. The kit of claim 78, wherein the paper is chromatography paper.

80. The kit of claim 79, wherein the chromatography paper is unmodified.
